(19) United States
(12) Patent Application Publication
(10) Pub. No.: US 2020/0071415 A1
(43) Pub. Date: Mar. 5, 2020

(54) COMBINATION THERAPY FOR PANCREATIC CANCER

(71) Applicant: ImClone LLC, Indianapolis, IN (US)

(72) Inventors: Damien Michel CRONIER, London (GB); Robert Leslie ILARIA, Jr., Zionsville, IN (US); Samuel Cowan RAMAGE, Indianapolis, IN (US)

(21) Appl. No.: 16/493,070

(22) PCT Filed: Mar. 9, 2018
(86) PCT No.: PCT/US2018/021724

§ 371 Date: Sep. 11, 2019

Related U.S. Application Data
(60) Provisional application No. 62/472,920, filed on Mar. 17, 2017.

(51) Int. Cl. C07K 16/30; A61K 31/337; A61K 31/7068; A61K 47/64; C07K 16/28; A61P 35/04

(52) U.S. Cl. CPC C07K 16/303; A61K 31/337; A61P 35/04; A61K 47/643; C07K 16/2863; A61K 31/7068

(57) ABSTRACT

The present invention provides for use in treating and methods of treating pancreatic cancer, in particular, metastatic pancreatic cancer, comprising olaratumab, in combination with gemcitabine and nab-paclitaxel.

The present invention relates to the field of medicine. The present invention relates to combinations of an antibody against platelet-derived growth factor receptor alpha (PDGFRα) and gemcitabine and albumin-bound paclitaxel (henceforth, nab-paclitaxel), for example the combination of olaratumab with gemcitabine and nab-paclitaxel, and to methods of using the combinations to treat cancer, particularly pancreatic cancer, more particularly, first line metastatic pancreatic cancer.

Platelet-derived growth factor receptor alpha, a cell surface receptor tyrosine kinase, is a glycoprotein of mesenchymal origin. PDGFRα/platelet-derived growth factor (PDGF) signaling has been shown to be an important signaling regulator in epithelial mesenchymal transition in several types of cancers, including pancreatic cancer (Li SH, et al., Mol. Cell. Biochem. 2013; 373(1-2): 217-27; Ekpe-Adewuyi I, et al. Oncotarget 2016; 7(50): 83684-83700). PDGFR/PDGF signaling creates an autocrine and paracrine signaling loop in pancreatic cancer that is an active participant in the formation of desmoplastic reaction as well as the activation of pancreatic stellate cells—which are hallmarks of pancreatic cancer and are important contributors to the pancreatic cancer microenvironment (Mahadevan and Von Hoff, Mol. Cancer. Ther. 2007; 6(4): 1186-1197). Across numerous cancers evaluated based on data from The Cancer Genome Atlas (TCGA) research, overexpression of PDGFRα in pancreatic tumors, relative to normal tissue, is approximately 40%. Though little to no overexpression of endogenous PDGFRα ligands (PDGF-A, -B, and -C) is observed in pancreatic cancer tumors, relative to normal pancreas tissue, expression levels in normal pancreas are relatively high, suggesting that PDGF ligands are present.

Olaratumab (LARTRUVO®, IMC-3G3) is a recombinant human immunoglobulin G subclass 1 (IgG1)-type monoclonal antibody that binds to PDGFRα and blocks interaction between PDGFRα and its ligands. In addition to blocking ligand-induced cell mitogenesis and receptor autophosphorylation, olaratumab inhibits ligand-induced phosphorylation of the downstream signaling molecules Akt and mitogen-activated proteinkinase (Loizos et al., Mol Cancer Ther. 2005; 4(3): 369-379.). Olaratumab, sequences thereof, and methods of making and using this antibody, including for the treatment of neoplastic diseases such as solid and non-solid tumors, are disclosed in WO 2006/138729.

Gemcitabine (CAS Registry No. 95058-81-4), a nucleoside metabolic inhibitor, is the active pharmaceutical ingredient in GEMZAR®, which has been approved: in combination with carboplatin, for the treatment of advanced ovarian cancer that has relapsed at least 6 months after completion of platinum—based therapy; in combination with paclitaxel, for first-line treatment of metastatic breast cancer after failure of prior anthracycline-containing adjuvant chemotherapy, unless anthracyclines were clinically contraindicated; in combination with cisplatin for the treatment of non-small cell lung cancer; and as a single agent for the treatment of pancreatic cancer. See the US and European labels of GEMZAR® (gemcitabine).

Nab-paclitaxel (CAS Registry No. 33069-62-4, a microtubule inhibitor, is a nanoparticle albumin-bound formulation of paclitaxel (ABRAXANE®), which has been approved for the treatment of: metastatic breast cancer, after failure of combination chemotherapy for metastatic disease or relapse within 6 months of adjuvant chemotherapy; locally advanced or metastatic non-small cell lung cancer (NSCLC), as first-line treatment in combination with carboplatin; and metastatic adenocarcinoma of the pancreas as first-line treatment, in combination with gemcitabine. See the US and European labels of ABRAXANE® (nab-paclitaxel).

Pancreatic cancer is the fourth leading cause of cancer-related death in the US alone. Pancreatic ductal adenocarcinoma and its variants account for more than 90% of pancreatic malignancies. Due to the difficulty in early detection, most patients with pancreatic cancer have advanced disease that has spread, or metastasized, to other parts of the body, thus limiting the number of patients who are able to seek surgical resection as a treatment option. Prognosis of metastatic pancreatic cancer after treatment with chemotherapeutic agents, alone or in combination, is often poor.

Unfortunately, a cure for pancreatic, unresectable pancreatic, metastatic pancreatic, and unresectable metastatic pancreatic cancer remains elusive and therapeutic options remain limited, with poor prognosis for patients; treatments for pancreatic and metastatic pancreatic cancer remain a continuing unmet clinical need. There exists a need for more and different therapies that may prove to be effective in treating pancreatic and metastatic pancreatic cancer.

Novel methods for use of the combination of olaratumab, gemcitabine and nab-paclitaxel to treat metastatic pancreatic cancer are presented herein.

Accordingly, the present invention provides olaratumab for use in simultaneous, separate, or sequential combination with gemcitabine and nab-paclitaxel in the treatment of pancreatic cancer in a patient.

More particularly, the pancreatic cancer is metastatic pancreatic cancer. More particularly, the pancreatic cancer is unresectable, metastatic pancreatic cancer.

More particularly, olaratumab is administered at a dose of about 15 mg/kg to about 25 mg/kg, gemcitabine is administered at a dose of about 1000 mg/m², and nab-paclitaxel is administered at a dose of about 125 mg/m². More particularly, olaratumab is administered at a dose of 15 mg/kg to 25 mg/kg, gemcitabine is administered at a dose of 1000 mg/m², and nab-paclitaxel is administered at a dose of 125 mg/m².

More particularly, olaratumab is administered at a dose of about 15 mg/kg. More particularly, olaratumab is administered at a dose of 15 mg/kg.

More particularly, olaratumab is administered at a dose of about 20 mg/kg. More particularly, olaratumab is administered at a dose of 20 mg/kg.

More particularly, olaratumab is administered at a dose of about 25 mg/kg. More particularly, olaratumab is administered at a dose of 25 mg/kg.

More particularly, each of olaratumab, gemcitabine and nab-paclitaxel is administered on days 1, 8, and 15 of a 28 day cycle.

More particularly, olaratumab is administered on days 1 and 15 of a 28-day cycle, and each of gemcitabine and nab-paclitaxel is administered on days 1, 8, and 15 of a 28 day cycle.

More particularly, each 15 mg (or about 15 mg) dose of olaratumab is administered on days 1, 8, and 15 of a 28 day cycle, and each of gemcitabine and paclitaxel is administered on days 1, 8, and 15 of a 28 day cycle.

More particularly, each 20 mg dose (or about 20 mg) of olaratumab is administered on days 1, 8, and 15 of a 28 day cycle, and each of gemcitabine and paclitaxel is administered on days 1, 8, and 15 of a 28 day cycle.

More particularly, each 20 mg (or about 20 mg) dose of olaratumab is administered on days 1 and 15 of a 28 day cycle, and each of gemcitabine and paclitaxel is administered on days 1, 8 and 15 of a 28 day cycle.

More particularly, each of 25 mg dose (or about 25 mg) of olaratumab is administered on days 1 and 15 of a 28 day cycle, and each of gemcitabine and paclitaxel is administered on days 1, 8 and 15 of a 28 day cycle.

More particularly, the patient is treated with an H1 antagonist prior to olaratumab administration. More particularly, the H1 antagonist is diphenhydramine. More particularly, the H1 antagonist (e.g., diphenhydramine) is administered 30-60 minutes prior to olaratumab administration. More particularly, H1 antagonist (e.g., diphenhydramine) is administered intravenously.

More particularly, the patient is treated with a corticosteroid prior to olaratumab administration. More particularly, the corticosteroid is selected from the group consisting of hydrocortisone, methylprednisolone, prednisolone, prednisone, triamcinolone, amcinonide, budesonide, desonide, fluocinolone acetonide, fluocinonide, halcinonide, triamcinolone acetonide, beclometasone, betametasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate and tixocortol pivalate. More particularly, the corticosteroid is dexamethasone. More particularly, the corticosteroid (e.g., dexamethasone) is administered 30-60 minutes prior to olaratumab administration. More particularly, the corticosteroid (e.g., dexamethasone) is administered intravenously.

The present invention also provides a method of treating pancreatic cancer in a patient, comprising administering simultaneously, separately, or sequentially to a patient in need of such a treatment an effective amount of each of olaratumab, gemcitabine and nab-paclitaxel.

In a preferred aspect, each of olaratumab, gemcitabine and nab-paclitaxel is administered by intravenous infusion.

More particularly, the pancreatic cancer is metastatic pancreatic cancer. More particularly, the pancreatic cancer is unresectable, metastatic pancreatic cancer.

More particularly, olaratumab is administered at a dose of about 15 mg/kg to about 25 mg/kg, gemcitabine is administered at a dose of about 1000 mg/m², and nab-paclitaxel is administered at a dose of about 125 mg/m². More particularly, olaratumab is administered at a dose of 15 mg/kg to 25 mg/kg, gemcitabine is administered at a dose of 1000 mg/m², and nab-paclitaxel is administered at a dose of 125 mg/m².

More particularly, olaratumab is administered at a dose of about 15 mg/kg. More particularly, olaratumab is administered at a dose of 15 mg/kg.

More particularly, olaratumab is administered at a dose of about 20 mg/kg. More particularly, olaratumab is administered at a dose of 20 mg/kg.

More particularly, olaratumab is administered at a dose of about 25 mg/kg. More particularly, olaratumab is administered at a dose of 25 mg/kg.

More particularly, each of olaratumab, gemcitabine and nab-paclitaxel is administered on days 1, 8, and 15 of a 28 day cycle.

More particularly, olaratumab is administered on days 1 and 15 of a 28-day cycle, and each of gemcitabine and nab-paclitaxel is administered on days 1, 8, and 15 of a 28 day cycle. In a preferred aspect, each of olaratumab, gemcitabine and nab-paclitaxel is administered by intravenous infusion.

More particularly, each 15 mg (or about 15 mg) dose of olaratumab is administered on days 1, 8, and 15 of a 28 day cycle, and each of gemcitabine and paclitaxel is administered on days 1, 8, and 15 of a 28 day cycle.

More particularly, each 20 mg dose (or about 20 mg) of olaratumab is administered on days 1, 8, and 15 of a 28 day cycle, and each of gemcitabine and paclitaxel is administered on days 1, 8, and 15 of a 28 day cycle.

More particularly, each 20 mg (or about 20 mg) dose of olaratumab is administered on days 1 and 15 of a 28 day cycle, and each of gemcitabine and paclitaxel is administered on days 1, 8 and 15 of a 28 day cycle.

More particularly, each of 25 mg dose (or about 25 mg) of olaratumab is administered on days 1 and 15 of a 28 day cycle, and each of gemcitabine and paclitaxel is administered on days 1, 8 and 15 of a 28 day cycle.

More particularly, the patient is treated with an H1 antagonist prior to olaratumab administration. More particularly, the H1 antagonist is diphenhydramine. More particularly, the H1 antagonist (e.g., diphenhydramine) is administered 30-60 minutes prior to olaratumab administration. More particularly, H1 antagonist (e.g., diphenhydramine) is administered intravenously.

More particularly, the patient is treated with a corticosteroid prior to olaratumab administration. More particularly, the corticosteroid is selected from the group consisting of hydrocortisone, methylprednisolone, prednisolone, prednisone, triamcinolone, amcinonide, budesonide, desonide, fluocinolone acetonide, fluocinonide, halcinonide, triamcinolone acetonide, beclometasone, betametasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate and tixocortol pivalate. More particularly, the corticosteroid is dexamethasone. More particularly, the corticosteroid (e.g., dexamethasone) is administered 30-60 minutes prior to olaratumab administration. More particularly, the corticosteroid (e.g., dexamethasone) is administered intravenously.

The present invention also provides the use of olaratumab, gemcitabine and nab-paclitaxel in the manufacture of a medicament for simultaneous, separate, or sequential treatment of pancreatic cancer.

More particularly, the pancreatic cancer is metastatic pancreatic cancer. More particularly, the pancreatic cancer is unresectable, metastatic pancreatic cancer.

More particularly, olaratumab is administered at a dose of about 15 mg/kg to about 25 mg/kg, gemcitabine is administered at a dose of about 1000 mg/m², and nab-paclitaxel is administered at a dose of about 125 mg/m². More particularly, olaratumab is administered at a dose of 15 mg/kg to 25 mg/kg, gemcitabine is administered at a dose of 1000 mg/m², and nab-paclitaxel is administered at a dose of 125 mg/m².

More particularly, olaratumab is administered at a dose of about 15 mg/kg. More particularly, olaratumab is administered at a dose of 15 mg/kg.

More particularly, olaratumab is administered at a dose of about 20 mg/kg. More particularly, olaratumab is administered at a dose of 20 mg/kg.

More particularly, olaratumab is administered at a dose of about 25 mg/kg. More particularly, olaratumab is administered at a dose of 25 mg/kg.

More particularly, each of olaratumab, gemcitabine and nab-paclitaxel is administered on days 1, 8, and 15 of a 28 day cycle.

More particularly, olaratumab is administered on days 1 and 15 of a 28-day cycle, and each of gemcitabine and nab-paclitaxel is administered on days 1, 8, and 15 of a 28 day cycle.

More particularly, each 15 mg (or about 15 mg) dose of olaratumab is administered on days 1, 8, and 15 of a 28 day cycle, and each of gemcitabine and paclitaxel is administered on days 1, 8, and 15 of a 28 day cycle.

More particularly, each 20 mg dose (or about 20 mg) of olaratumab is administered on days 1, 8, and 15 of a 28 day cycle, and each of gemcitabine and paclitaxel is administered on days 1, 8, and 15 of a 28 day cycle.

More particularly, each 20 mg (or about 20 mg) dose of olaratumab is administered on days 1 and 15 of a 28 day cycle, and each of gemcitabine and paclitaxel is administered on days 1, 8 and 15 of a 28 day cycle.

More particularly, each of 25 mg dose (or about 25 mg) of olaratumab is administered on days 1 and 15 of a 28 day cycle, and each of gemcitabine and paclitaxel is administered on days 1, 8 and 15 of a 28 day cycle.

In a preferred aspect, each of olaratumab, gemcitabine and nab-paclitaxel is administered by intravenous infusion.

More particularly, the patient is treated with an H1 antagonist prior to olaratumab administration. More particularly, the H1 antagonist is diphenhydramine. More particularly, the H1 antagonist (e.g., diphenhydramine) is administered 30-60 minutes prior to olaratumab administration. More particularly, H1 antagonist (e.g., diphenhydramine) is administered intravenously.

More particularly, the patient is treated with a corticosteroid prior to olaratumab administration. More particularly, the corticosteroid is selected from the group consisting of hydrocortisone, methylprednisolone, prednisolone, prednisone, triamcinolone, amcinonide, budesonide, desonide, fluocinolone acetonide, fluocinonide, halcinonide, triamcinolone acetonide, beclometasone, betametasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate and tixocortol pivalate. More particularly, the corticosteroid is dexamethasone. More particularly, the corticosteroid (e.g., dexamethasone) is administered 30-60 minutes prior to olaratumab administration. More particularly, the corticosteroid (e.g., dexamethasone) is administered intravenously.

The present invention also provides a kit comprising a container comprising a pharmaceutical composition comprising olaratumab with one or more pharmaceutically acceptable carriers, diluents, or excipients, a container comprising a pharmaceutical composition comprising gemcitabine with one or more pharmaceutically acceptable carriers, diluents, or excipients, and a container comprising a pharmaceutical composition comprising nab-paclitaxel with one or more pharmaceutically acceptable carriers, diluents, or excipients, for use in the treatment pancreatic cancer.

More particularly, the cancer is metastatic pancreatic cancer. More particularly, the cancer is unresectable metastatic pancreatic cancer.

More particularly, the kit comprises either (i) written instructions for administering olaratumab, gemcitabine and nab-paclitaxel, or (ii) an internet address(es) from which instructions for administering olaratumab, gemcitabine and nab-paclitaxel can be obtained.

In a preferred aspect, olaratumab, gemcitabine, and nab-paclitaxel are administered by intravenous infusion.

In one aspect of the invention, “olaratumab” refers to any antibody that is olaratumab as defined by the International Nonproprietary Name (INN) found in WHO Drug Information Vol. 25, No. 1, 2011, pp. 76-77. It has also been identified as IMC-3G3, and CAS registry number 1024603-93-7.

As used herein, “about” means±5% (e.g., 95-105 mg per week or 95-105 mg every other week).

As used herein, the terms “treating,” “to treat,” or “treatment” refers to restraining, slowing, stopping, reducing, or reversing the progression or severity of an existing symptom, disorder, condition, disease, or cancer.

As used herein, the term “patient” refers to a mammal, preferably a human.

As used herein, the term “kit” refers to a package comprising containers, e.g., vials. A “kit” may also include instructions to administer all or a portion of the contents of the containers to a cancer patient.

A potential advantage of the combination treatments of the invention is the possibility of producing marked and/or prolonged anti-cancer effects in a patient with an acceptable safety profile, including acceptable tolerability, toxicities and/or adverse events, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. Without being bound by theory, the therapeutic agents used in the invention may function by a variety of mechanisms, including an anti-tumor or anti-tumoristatic effect. Because the invention relates to the use of a unique combination of anti-tumor agents, various approaches to determining efficacy of any particular combination therapy of the present invention can be optionally employed, including, for example, cell-cycle dependent biomarkers measurement/visualization, and measurement of response through radiological imaging.

As used herein, the term “Complete Response” (CR) is defined as the disappearance of all target lesions. Any pathological lymph nodes (whether target or non-target) must have reduction in short axis to<10 mm. Tumor marker results must have normalized.

As used herein, the term “Partial Response” (PR) is defined as at least a 30% decrease in the sum of diameter of target lesions, taking as reference the baseline sum diameters.

As used herein the term “Progressive Disease” (PD) is defined as at least a 20% increase in the sum of the diameters of target lesions, taking as reference the smallest sum on study (including the baseline sum if that is the smallest). In addition to the relative increase of 20%, the sum must also demonstrate an absolute increase of at least 5 mm. The appearance of 1 or more new lesions is also considered progression. For equivocal findings of progression (for example, very small and uncertain new lesions; cystic changes or necrosis in existing lesions), treatment may continue until the next scheduled assessment. If at the next scheduled assessment, progression is confirmed, the date of progression should be the earlier date when progression was suspected.

As used herein, the term “Stable Disease” (SD) is defined as neither sufficient shrinkage to qualify for PR nor sufficient increase to qualify for PD, taking as reference the smallest sum diameters while on study.

As used herein, the term “Not Evaluable” (NE) is define as when an incomplete radiologic assessment of target lesions is performed or there is a change in the method of measurement from baseline that impacts the ability to make a reliable evaluation of response.

As used herein, the term “Progression-Free Survival” (PFS) is defined as the time from the date of first dose of any study drug until the date of radiographically documented PD or death due to any cause, whichever is earlier.

As used herein, the term “Overall Survival” (OS) is defined as the time from the date of study first dose of any study drug to the date of death from any cause.

As used herein, the term “Duration of Response” is defined as the time from the date measurement criteria for CR or PR (whichever is first recorded) are first met until the first date that disease is recurrent or objective progression is observed, per RECIST 1.1 criteria, or the date of death from any cause in the absence of objectively determined disease progression or recurrence.

As used herein, the term “Disease Control Rate” (DCR) is defined as the proportion of patients achieving a best overall response of SD, PR, or CR.

As used herein, the term “Objective Response Rate” is defined as the proportion of patients achieving a best overall response of PR or CR from randomization until PD/recurrence divided by the total number of patients randomized to the corresponding treatment arm (ITT population). As used herein, the term “Overall Response Rate” is based on each patient's best objective response and will be determined for all patients evaluable via the Response Evaluation Criteria in Solid Tumors (RECIST) v 1.1 criteria. The Overall Response Rate (%) will be calculated as the number of patients with best objective response of CR or PR divided by the number of patients with measurable disease at baseline. The best objective response for a given patient will be based on objective responses determined from data obtained up to: progression or the last evaluable assessment in the absence of progression. Patients for whom an objective response cannot be determined, or for who the best objective response is NE, will be considered non-responders. The Overall Response Rate will be summarized along with the 95% Clopper Pearson confidence interval.

As used herein, the term “effective amount” refers to the amount or dose of olaratumab and/or to the amount or dose of gemcitabine and nab-paclitaxel, which, upon single or multiple dose administration to the patient, provides an effective response in the patient under diagnosis or treatment

As used herein, the term “effective response” of a patient or a patient's “responsiveness” to treatment with a combination of agents and similar wording refers to the clinical or therapeutic benefit imparted to a patient upon co-administration of gemcitabine and nab-paclitaxel, and olaratumab. Such benefit includes any one or more of: extending survival (including OS and PFS); resulting in an objective response (including a CR or a PR); or improving signs or symptoms of cancer, etc.

As used herein, intravenous infusion and intravenous injection can be used interchangeably.

As used herein, the phrase “in combination with” refers to the simultaneous, separate, or sequential administration of olaratumab with gemcitabine and nab-paclitaxel. As used herein, the phrase “in combination with” also refers to the administration of olaratumab with gemcitabine and nab-paclitaxel sequentially in any order. As used herein, the phrase “in combination with” also refers to the administration of olaratumab with gemcitabine and nab-paclitaxel in any combination thereof. Gemcitabine and nab-paclitaxel can be administered prior to administration of olaratumab. Gemcitabine and nab-paclitaxel can be administered at the same time as administration of olaratumab. Gemcitabine and nab-paclitaxel can be administered subsequent to administration of olaratumab. Gemcitabine and nab-paclitaxel can be administered prior to, at the same time as, or subsequent to administration of olaratumab, or in some combination thereof.

Where olaratumab is administered at repeated intervals (e.g., during a standard course of treatment), gemcitabine and nab-paclitaxel can be administered prior to each administration of olaratumab. Where olaratumab is administered at repeated intervals (e.g,. during a standard course of treatment), gemcitabine and nab-paclitaxel can be administered at the same time as each administration of olaratumab. Where olaratumab is administered at repeated intervals (e.g,. during a standard course of treatment), gemcitabine and nab-paclitaxel can be administered subsequent to each administration of olaratumab. Where olaratumab is administered at repeated intervals (e.g., during a standard course of treatment), gemcitabine and nab-paclitaxel can be administered prior to, at the same time as, or subsequent to, each administration of olaratumab or some combination thereof. Where olaratumab is administered at repeated intervals (e.g., during a standard course of treatment), gemcitabine and nab-paclitaxel can be administered at different intervals in relation to therapy with olaratumab. Where olaratumab is administered at repeated intervals (e.g., during a standard course of treatment), gemcitabine and nab-paclitaxel can be administered in a single or series of dose(s) prior to, at any time during, or subsequent to the course of treatment with olaratumab. Where olaratumab is administered at repeated intervals (e.g., during a standard course of treatment), gemcitabine and nab-paclitaxel can be administered in single or sequential doses prior to, at any time during, or subsequent to the course of treatment with olaratumab. Where olaratumab is administered at repeated intervals (e.g. during a standard course of treatment), gemcitabine and nab-paclitaxel can be administered in single or sequential doses prior to the course of treatment with olaratumab. Where olaratumab is administered at repeated intervals (e.g., during a standard course of treatment), gemcitabine and nab-paclitaxel can be administered in single or sequential doses at any time during the course of treatment with olaratumab. Where olaratumab is administered at repeated intervals (e.g., during a standard course of treatment), gemcitabine and nab-paclitaxel can be administered in single or sequential doses subsequent to the course of treatment with olaratumab. Where olaratumab is administered at repeated intervals (e.g., during a standard course of treatment), gemcitabine and nab-paclitaxel can be administered in a series of doses prior to the course of treatment with olaratumab. Where olaratumab is administered at repeated intervals (e.g., during a standard course of treatment), gemcitabine and nab-paclitaxel can be administered in a series of doses subsequent to the course of treatment with olaratumab. Where olaratumab is administered at repeated intervals (e.g., during a standard course of treatment), gemcitabine and nab-paclitaxel can be administered in a series of doses subsequent to the course of treatment with olaratumab.

The following examples and clinical study results further illustrate the present invention.

The following clinical study designs further illustrate the invention, but should not be construed to limit the scope of the invention in any way.

### A STUDY OF OLARATUMAB IN COMBINATION WITH GEMCITABINE AND NAB-PACLITAXEL IN THE FIRST-LINE TREATMENT OF METASTATIC PANCREATIC CANCER

### Clinical Study Design

Open label, randomized, double-blinded Phase 1b/Phase 2 study (hereinafter “Study”) to investigate the effectiveness, safety, and tolerability of olaratumab combined with gemcitabine and nab-paclitaxel in˜186 patients with Stage IV unresectable metastatic pancreactic cancer who have not received prior treatment for metastatic disease. The Study consists of several parts including:
Part 1: dose escalation of olaratumab (15 mg/kg and 20 mg/kg IV) are administered on Day 1, Day 8, and Day 15 every 28 days in combination with gemcitabine (1000 mg/m² IV) and Nab-paclitaxel (125 mg/m² IV) or dose escalation of olaratumab (20 mg/kg and 25 mg/kg IV) on Day 1 and Day 15 every 28 days in patients with Stage IV metastatic pancreatic cancer.
Part 2: expansion cohort with the dose and schedule of olaratumab identified in Part 1 in combination with a fixed regimen of gemcitabine (1000 mg/m² IV) and Nab-paclitaxel (125 mg/m² IV) on Day 1, Day 8, and Day 15 every 28 days in patients with Stage IV metastatic pancreatic cancer.

Approximately 24 patients are enrolled in Part 1, with between 3 to 6 patients enrolled at each dose level of olaratumab, to identify the minimum tolerated dose (MTD). After MTD is established, a cohort expansion of 9 additionally enrolled patients is performed to further evaluate MTD. After determination of the MTD in the dose escalation portion of the study and confirmation of the safety and tolerability of that dose in the cohort expansion, a review of safety data, including the number and type of dose-limiting toxicities (DLTs), other safety information, and relevant PK, is conducted prior to proceeding to Part 2.

Approximately 162 patients are enrolled in Part 2 in a randomized, double-blinded 2-arm study of olaratumab with and without a fixed regimen of gemcitabine (1000 mg/m2 IV) and Nab-paclitaxel (125 mg/m2 IV) on Day 1, Day 8, and Day 15 every 28 days. Patients will receive the study treatment until disease progression or a criterion for discontinuation is met. The primary end point is OS; secondary end points are PFS, duration of response (DoR), objective response rate (ORR), patient-reported outcomes (PROs), and safety.

Patients are assessed for tumor response every 8 weeks. Patients without disease progression may continue to receive treatment until the development of unacceptable toxicity, death, or other discontinuation criteria are met. Study completion is expected approximately 6-12 months after the last patient has been enrolled.

Major eligibility criteria include: histological or cytological diagnosis of metastatic adenocarcinoma of the exocrine pancreas; measurable or nonmeasurable but evaluable disease as defined by the Response Evaluation Criteria in Solid Tumors (RECIST 1.1, Eisenhauer et al., 2009); a performance status (PS) of 0 to 1 on the Eastern Cooperative Oncology Group (ECOG) scale (Oken et al., 1982); and no prior systemic treatment for metastatic disease.

### Study Objectives

The primary objective of Part 1 of the Study is to investigate safety and tolerability of olaratumab administered at the doses of 15 mg/kg and 20 mg/kg IV on days 1, 8 and 15, and 20 mg/kg and 25 mg/kg IV on Days 1 and 15, of 28-day cycles in patients with Stage IV pancreatic cancer with gemcitabine (1000 mg/m² IV on Days 1, 8, and 15) and nab-paclitaxel (125 mg/m² IV on Days 1, 8, and 15) as measured by number of patients with a DLT during Cycle 1; and for Part 2 to evaluate the efficacy of olaratumab in combination with or without gemcitabine and nab-paclitaxel in terms of OS in patients with Stage IV pancreatic cancer.

The secondary objectives of the Study include: for Part 1: (i) to characterize the safety and toxicity profile of olaratumab plus gemcitabine and nab-paclitaxel; (ii) to evaluate the PK and immunogenicity of olaratumab plus gemcitabine and nab-paclitaxel; and (iii) to document the antitumor activity observed with olaratumab plus gemcitabine and nab-paclitaxel; for Part 2: (iv) to assess time-to-event variables; (v) to document the antitumor activity observed with olaratumab plus gemcitabine and nab-paclitaxel; and (vi) to assess the PK and immunogenicity of olaratumab plus gemcitabine and nab-paclitaxel. The correlation of potential biomarkers to clinical outcome may also be assessed.

In the Part 1 portion of the study, doses of olaratumab and/or nab-paclitaxel or gemcitabine may be delayed, omitted, or reduced if the patient experiences an adverse event or a DLT-equivalent toxicity.

In the Part 2 portion, investigators are be blinded to treatment assignment (olaratumab versus placebo) and should adjust dosing of olaratumab/placebo as if all patients are receiving olaratumab.

In the event of an alteration in olaratumab/placebo dose due to an olaratumab/placebo-related toxicity, gemcitabine or nab-paclitaxel need not be altered, and the planned gemcitabine or Nab-paclitaxel schedule is maintained. Similarly, olaratumab/placebo therapy need not be altered for either gemcitabine- or nab-paclitaxel-related toxicity.

Omission or discontinuation of one of the study drugs in the combination does not prohibit dosing with other study drugs, provided the patient meets dosing criteria.

In case of difficulty in assigning relatedness to one study drug or the other, the doses of all study drugs may be delayed, reduced, or omitted. Treatment may be delayed for up to 21 days to allow a patient sufficient time for recovery from study treatment-related toxicity.

### Findings

As of Feb. 15, 2018, the study is currently ongoing with multiple patients being treated under Part 1 of the protocol. A well tolerated safety profile with no treatment related serious adverse reactions has been observed thus far.

We claim:

1-13. (canceled)

14. A method of treating pancreatic cancer in a patient, comprising administering simultaneously, separately, or sequentially to a patient in need of such a treatment an effective amount of each of olaratumab, gemcitabine, and nab-paclitaxel.

15. The method of claim 14, wherein the pancreatic cancer is metastatic pancreatic cancer.

16. The method of claim 15, wherein the metastatic pancreatic cancer is unresectable, metastatic pancreatic cancer.

17. The method of claim 14, wherein olaratumab is administered at a dose of about 15 mg/kg to about 25 mg/kg, wherein gemcitabine is administered at a dose of about 1000 mg/m², and wherein nab-paclitaxel is administered at a dose of about 125 mg/m².

18. The method of claim 17, wherein olaratumab is administered at a dose of about 15 mg/kg, wherein gemcitabine is administered at a dose of about 1000 mg/m², and wherein nab-paclitaxel is administered at a dose of about 125 mg/m².

19. The method of claim 18, wherein each of olaratumab, gemcitabine, and nab-paclitaxel is administered on days 1, 8, and 15 of a 28-day cycle.

20. The method of claim 17, wherein olaratumab is administered at a dose of about 20 mg/kg, wherein gemcitabine is administered at a dose of about 1000 mg/m², and wherein nab-paclitaxel is administered at a dose of about 125 mg/m².

21. The method of claim 20, wherein each of olaratumab, gemcitabine, and nab-paclitaxel is administered on days 1, 8, and 15 of a 28-day cycle.

22. The method of claim 20, wherein olaratumab is administered on days 1 and 15 of a 28-day cycle, and each of gemcitabine and nab-paclitaxel is administered on days 1, 8, and 15 of a 28-day cycle.

23. (canceled)

24. The method of claim 17, wherein olaratumab is administered at a dose of about 25 mg/kg, wherein gemcitabine is administered at a dose of about 1000 mg/m², and wherein nab-paclitaxel is administered at a dose of about 125 mg/m².

25. (canceled)

26. The method of claim 24, wherein olaratumab is administered on days 1 and 15 of a 28-day cycle, and each of gemcitabine and nab-paclitaxel is administered on days 1, 8, and 15 of a 28-day cycle.

27. A kit comprising a container comprising a pharmaceutical composition comprising olaratumab with one or more pharmaceutically acceptable carriers, diluents, or excipients, a container comprising a pharmaceutical composition comprising gemcitabine with one or more pharmaceutically acceptable carriers, diluents, or excipients, and a container comprising a pharmaceutical composition comprising nab-paclitaxel with one or more pharmaceutically acceptable carriers, diluents, or excipients, for use in the treatment pancreatic cancer.

28. The kit of claim 27, further comprising either (i) written instructions for administering olaratumab, gemcitabine, and nab-paclitaxel, or (ii) an internet address from which instructions for administering olaratumab, gemcitabine, and nab-paclitaxel can be obtained.

29-41. (canceled)
